(19) United States
(12) Patent Application Publication
(10) Pub. No.: US 2006/0252731 A1
(43) Pub. Date: Nov. 9, 2006

(54) Methods of treating recurrent aphthous stomatitis

(76) Inventors: David F. Pfeiffer, Blue Bell, PA (US); Christopher Powala, Radnor, PA (US)

Correspondence Address: HOFFMANN & BARON, LLP, 6900 JERICHO TURNPIKE, SYOSSET, NY, 11791, US

(21) Appl. No.: 11/124,612

(22) Filed: May 6, 2005

(51) Int. Cl. A61K 31/65

(52) U.S. Cl. 514/152

(57) ABSTRACT

A method of treating recurrent aphthous stomatitis in a human in need thereof. The method comprises administering a tetracycline compound in an amount that is effective to treat recurrent aphthous stomatitis, but has substantially no antibacterial activity, without administering a bisphosphonate compound.

### BACKGROUND OF THE INVENTION

Recurrent aphthous stomatitis (RAS) is a common condition characterized by recurring oval or round ulcerative lesions on the oral mucosa. These ulcerative lesions are known as aphthous ulcers and, more commonly, as canker sores. Aphthous ulcers are typically accompanied by inflammation and substantial pain, especially upon eating, swallowing and speaking. It has been estimated that about 20% of the general population has been afflicted with RAS at some time (Ylikontiola et al., Oral Surg., Oral Med., Oral Path., Oral Radiol. Endod. 83:329-333 (1997).)

Distinguishing characteristics of the ulcers of RAS include their chronic nature, their location, and their appearance. These ulcers typically form on the loose tissues of the oral mucosa, i.e., on unkeratinized oral mucosal surfaces. Examples of such tissues include the inside of the lips and cheeks, the floor of the mouth, the underside of the tongue, the soft palate, and the tonsillar areas. The tissues surrounding the ulcers appear healthy and the patient usually does not have systemic features (such as a fever or malaise). Generally, RAS has three clinical presentations as discussed below.

Minor aphthae, also referred to as Mikulicz's aphthae or mild aphthous ulceration, account for 75 to 85 percent of all cases of RAS. Minor aphthae can involve every nonkeratinized mucosa of the oral cavity, are smaller than 8 to 10 mm and tend to heal within 10 to 14 days without scarring.

Major aphthae, also referred as periadenitis mucosa necrotica recurrens or Sutton's disease, tend to be localized to mucosa overlying minor salivary glands. Approximately 10 to 15 percent of RAS cases are major aphthae. Ulcers associated with major aphthae have raised irregular borders and frequently exceed one centimeter in diameter. Compared with the ulcers of minor aphthae, the ulcers of major aphthae are deeper, larger, more painful, and last significantly longer. In particular, these ulcers can last for weeks or months and often leave a scar after healing. Fever, dysphagia and malaise sometimes occur early in the disease process of major aphthae.

Herpetiform ulcers constitute about 5 to 10% of RAS cases. Multiple one to three millimeter crops of small, rounded, painful ulcers, resembling ulcers of herpes simplex, appear anywhere on the mucosa. These small ulcers tend to fuse and produce larger ulcers lasting ten to fourteen days.

Etiology

The etiology of RAS is unknown. A wide variety of etiologies for RAS has been proposed, such as microbial, genetic, autoimmune, allergic and idiopathic.

Although the etiology of RAS is undetermined, several factors seem to be associated with, and/or precipitate, aphthous ulcers in predisposed individuals. Such factors include, for example, emotional stress, prolonged fever, food or drug hypersensitivity, hormonal changes, infections, immunodeficiency and vitamin deficiencies.

Treatment

Generally, the treatment of RAS has been palliative, including various measures to lessen the pain, limit the duration, and reduce the inflammatory reaction of the aphthous ulcers. The types of treatments include the use of over-the-counter and prescription medications that have been specially formulated for the treatment of aphthous ulcers. These treatments have met with only limited success.

Some of these medications create a protective barrier film over aphthous ulcers. Such barrier films help to reduce the ulcer's exposure to irritating substances such as food and drink. However, these films do not per se heal, or speed the healing of, ulcers.

Additionally, some products contain numbing agents (such as benzocaine, benzoin tincture, lidocaine, camphor, and phenol) that minimize the degree to which aphthous ulcers interfere with normal daily activities. Typically, these products are indicated for short-term usage only.

Anti-inflammatory medications have also been used in the treatment of aphthous ulcers. Some examples of topically applied anti-inflammatory medications include amlexanox; and corticosteroids, such as triamcinolone acetonide, fluocinonide, betamethasone and clobetasol. Research has demonstrated that the use of these medications can alleviate the pain from aphthous ulcers, accelerate their healing, and limit the extent to which an ulcer will progress. However, these medications have not been shown to reduce the rate of recurrence of aphthous ulcers. Additionally, a problem associated with topical corticosteriods is that they may facilitate the overgrowth of Candida.

A variety of microbes has been isolated from aphthous ulcers. Accordingly, suppression or elimination of microbes that populate such ulcers is one of the conventional treatment approaches to RAS.

For example, antimicrobial mouthwashes have been prescribed to treat aphthous ulcers. Such mouthwashes are applied topically, and typically include one or more of thymol, copper sulfate, iodine, chlorhexidine gluconate and tetracycline. Use of such mouthwashes several times a day has been shown to reduce the pain associated with aphthous ulcers and also to speed up their healing. However, the use of these washes has not been shown to reduce the rate of recurrence of the ulcers.

In addition to inhibiting the microbial population inaphthous ulcers, it has been proposed that antimicrobial tetracycline mouthwashes inhibit salivary collagenase levels. This proposal was investigated by studying the salivary collagenase levels in RAS patients who rinsed their mouths with chlortetracycline (Hayrinen-Immonen et al., J. Oral Pathol. Med. 23:269-72 (1994)). Although the rinsing alleviated the discomfort caused by aphthous ulcers, no reduction in the in vivo salivary collagenase levels was observed.

It has also been suggested that, in order to effectively treat aphthous ulcers, it is necessary to contact the ulcers with a “supratherapeutic” concentration of antibacterial agent(s). See U.S. Pat. No. 6,248,718. In this patent, a “supratherapeutic” concentration of antibacterial agent(s) is provided in the form of a topically-applied lozenge containing antibacterial agent(s). It was reported that the use of this lozenge resulted in the reduction of ulcer pain in about forty-eight hours, and the healing of ulcers in about four days.

Other methods used to keep medications in contact with ulcers for an extended period of time include the use of tissue adhesives. Doxycycline applied with cyano-acrylate adhesive was found to decrease the intensity of ulcer pain (Ylikontiola et al., Oral Surg. Oral Med. Oral Path. 83:329-33 (1997)).

Additionally, continuous systemic treatment with antimicrobial amounts of tetracycline and doxycycline has been reported to achieve long remission phases of aphthous ulcers (Conklin et al., Intl. J. Dermatol. 30(5):323-35 (1991)). The mode of action of such treatment has been reported as being mainly antimicrobial (Martindale. The Extra Pharmacopoeia, p. 1220 (James E. F. Reynolds ed., 28^{th} ed. 1982)).

However, the systemic use of antimicrobial amounts of tetracyclines can lead to undesirable side effects. For example, the long term administration of antimicrobial tetracyclines can reduce or eliminate healthy microbial flora, such as intestinal flora, and can lead to the production of antimicrobial resistant organisms or the overgrowth of yeast and fungi.

Accordingly, there is a need for an effective treatment of RAS. There is a particular need for a treatment which reduces the recurrence rate of aphthous ulcers while causing fewer undesirable side effects.

### SUMMARY OF THE INVENTION

The present invention is a method of treating recurrent aphthous stomatitis in a human in need thereof. The method comprises administering a tetracycline compound in an amount that is effective to treat recurrent aphthous stomatitis, but has substantially no antibacterial activity, without administering a bisphosphonate compound.

### BRIEF DESCRIPTION OF DRAWINGS

FIG. 1 is a graph of the results of the Visual Analogue Scale indicating the amount of pain associated with ulcers.

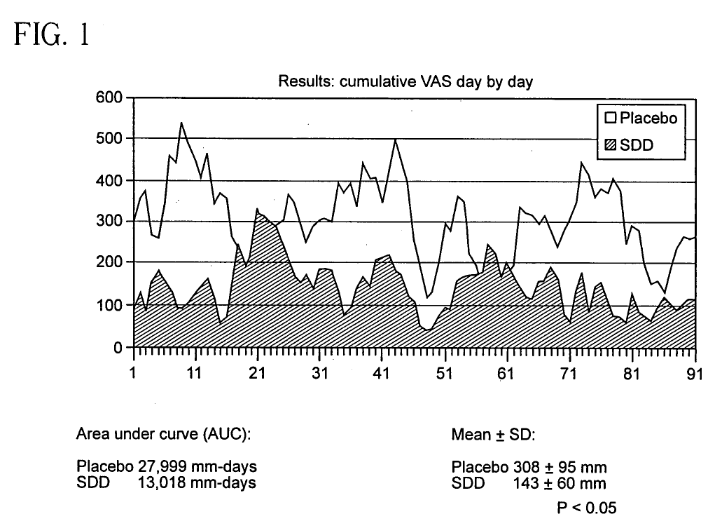

FIG. 2 is a graph quantifying the appearance of new ulcers in the participants of the clinical study.

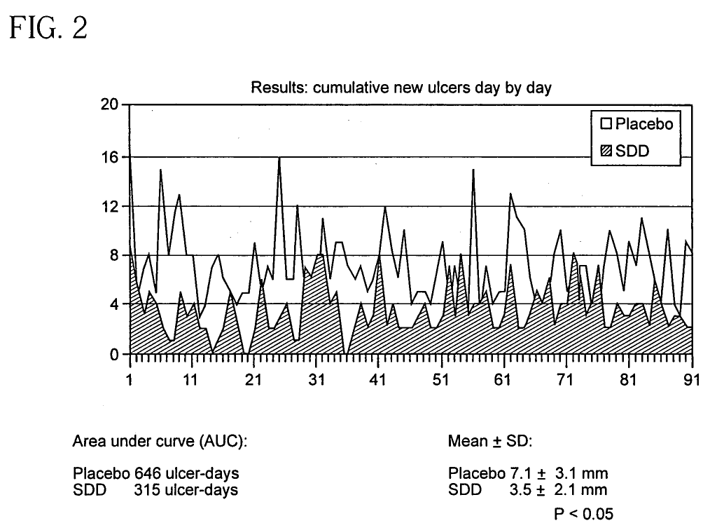

### DETAILED DESCRIPTION OF THE INVENTION

The present invention provides a method for treating recurrent aphthous stomatitis (RAS). As used herein, the term “recurrent aphthous stomatitis” is a chronic disorder characterized by recurring oval or round ulcerative lesions presenting on the oral mucosa. These ulcers are typically accompanied by inflammation and substantial pain.

The present invention is effective in treating all types of RAS. Some types of RAS include, for example, minor aphthae, Mikulicz's aphthae, mild aphthous ulceration, major aphthae, periadenitis mucosa necrotica recurrens, Sutton's disease and herpetiform ulcers.

The method of the present invention comprises the administration of a tetracycline compound to a human in an amount which is effective for the treatment of RAS, but which has substantially no antibacterial activity, without administering a bisphosphonate compound. Preferably, the human is monitored. Monitoring is accomplished by observing a positive result. A positive result includes reducing or reversing the characteristics of RAS, such as, for example, reducing the recurrence, size and/or discomfort of the symptomatic lesions. After a positive result is observed, treatment is continued.

The tetracycline compound can be an antibacterial or non-antibacterial compound. The tetracyclines are a class of compounds of which tetracycline is the parent compound. Tetracycline has the following general structure: 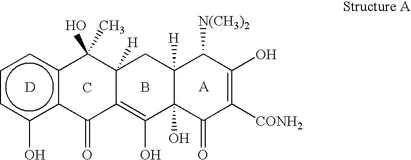
The numbering system of the multiple ring nucleus is as follows:

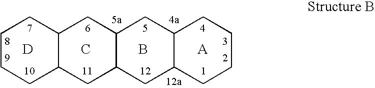

Tetracycline, as well as the 5-hydroxy (oxytetracycline, e.g., Terramycin) and 7-chloro (chlorotetracycline, e.g., Aureomycin) derivatives, exist in nature, and are all well known antibacterials. Semisynthetic derivatives such as 7-dimethylaminotetracycline (minocycline) and 6α-deoxy-5-hydroxytetracycline (doxycycline) are also known tetracycline antibacterials. Natural tetracyclines may be modified without losing their antibacterial properties, although certain elements of the structure must be retained to do so.

Some examples of antibacterial (i.e., antimicrobial) tetracycline compounds include doxycycline, minocycline, tetracycline, oxytetracycline, chlortetracycline, demeclocycline, lymecycline and their pharmaceutically acceptable salts.

Non-antibacterial tetracycline compounds are structurally related to the antibacterial tetracyclines, but have had their antibacterial activity substantially or completely eliminated by chemical modification. For example, non-antibacterial tetracycline compounds are at least about ten times, preferably at least about twenty five times, less antibacterial than doxycycline. In other words, non-antibacterial tetracycline compounds are incapable of achieving antibacterial activity comparable to that of doxycyline at concentrations at least about ten times, preferably at least about twenty five times, greater than that of doxycycline.

Examples of chemically modified non-antibacterial tetracyclines include 4-de(dimethylamino)tetracycline (COL-1), tetracyclinonitrile (COL-2), 6-demethyl-6-deoxy-4-de(dimethylamino)tetracycline (COL-3), 7-chloro-4-de(dimethylamino)-tetracycline (COL-4), tetracycline pyrazole (COL-5), 4-hydroxy-4-de(dimethylamino)-tetracycline (COL-6), 4-de(dimethylamino-12α-deoxytetracycline (COL-7), 6-deoxy-5α-hydroxy-4-de(dimethylamino)tetracycline (COL-8), 4-de(dimethylamino)-12α-deoxyanhydrotetracycline (COL-9), and 4-de(dimethylamino)minocycline (COL-10).

Tetracycline derivatives, for purposes of the invention, may be any tetracycline derivative, including those compounds disclosed generically or specifically in U.S. Pat. No. 6,638,922 issued on Oct. 28, 2003 (assigned to CollaGenex Pharmaceuticals, Inc. of Newtown, Pa.) which are herein incorporated by reference.

Throughout this specification, parameters are defined by maximum and minimum amounts. Each minimum amount can be combined with each maximum amount to define a range.

According to the present invention, a tetracycline compound is administered in a sub-antibacterial amount. A sub-antibacterial amount of a tetracycline compound is any amount that results in a tetracycline plasma concentration: (i) which is effective to treat recurrent aphthous stomatitis (RAS), but (ii) which has no, or substantially no, antibacterial activity. A treatment is effective if it causes one or more of: (i) a reduction or inhibition of the ulcers associated with RAS; (ii) a reduction in the recurrence rate of aphthous ulcers; and (iii) a reduction or inhibition of the pain, discomfort and/or inflammation associated with the ulcers.

A concentration of a tetracycline compound having substantially no antibacterial activity is any concentration that does not significantly prevent the growth of bacteria. That is, a microbiologist would not consider the growth of bacteria to be inhibited from a clinical point of view.

In one embodiment, the tetracycline compound is an antibacterial tetracycline compound. One way in which to quantify the antibacterial activities of tetracyclines is by a measure called minimum inhibitory concentration (MIC), as is known by a skilled artisan.

An MIC is the minimum tetracycline concentration that inhibits the growth of a particular strain of bacteria in vitro. MIC values are determined using standard procedures. Standard procedures are, for example, based on a dilution method (broth or agar), or an equivalent, using standard concentrations of inoculum and tetracycline powder. See, for example, National Committee for Clinical Laboratory Standards. Performance Standards for Antimicrobial Susceptibility Testing—Eleventh Informational Supplement. NCCLS Document M100-S11, Vol. 21, No. 1, NCCLS, Wayne, Pa., January, 2001.

In order to inhibit the growth of a strain of bacteria in vivo, a tetracycline compound achieves a plasma concentration in excess of the MIC for the strain. Plasma concentration refers to the concentration of a tetracycline compound measured in an individual's blood sample taken at steady state. Steady state is generally achieved after dosing for five to seven terminal half lives. The half lives of different tetracycline compounds vary from hours to days.

In the present invention, a tetracycline compound is administered in an amount that is effective, as described above, and that results in a plasma concentration which is significantly below the MIC for commonly-occurring bacteria. Such amounts are considered to have no, or substantially no, antibacterial activity. Examples of commonly-occurring bacteria that are susceptible to tetracyclines are Escherichia coli (e.g., ATCC 25922 and 25922); Neisseria gonorrhoeae (e.g., ATCC 49226); Staphylococcus aureus (e.g., ATCC 29213 and 25213); and Streptococcus pneumoniae (e.g., ATCC 49619).

For example, in the present invention, a tetracycline compound is administered in an amount that results in a plasma concentration which is less than approximately 80%, 75%, 70%, 65%, 60%, 55%, 50%, 45%, 40%, 35%, 30%, 25%, 20%, 15%, 10%, 5%, 1% or 0.5% of the MIC for the commonly-occurring bacteria mentioned above. A skilled artisan can readily determine the amount of a particular tetracycline compound to administer to achieve such concentrations.

For example, doxycycline is administered in an amount that results in a minimum steady state plasma concentration of about 0.1 μg/ml, 0.2 μg/ml, or 0.3 μg/ml, and a maximum steady state plasma concentration of about 0.7 μg/ml, 0.8 μg/ml, or 0.9 μg/ml.

The sub-antibacterial amount of an antibacterial tetracycline compound can also be expressed by daily dose. The daily dose of an antibacterial tetracycline compound is any amount that is sufficient to produce the effective, sub-antibacterial plasma concentrations described above. Such dose can, for example, be expressed as a percentage of a minimum antibacterial daily dose.

A skilled artisan knows, or is able routinely to determine, the minimum antibacterial daily dose for tetracycline compounds. Examples of suitable sub-antibacterial doses of antibacterial tetracycline compounds for the treatment of RAS include less than approximately: 80%, 75%, 70%, 65%, 60%, 55%, 50%, 45%, 40%, 35%, 30%, 25%, 20%, 15%, 10%, 5%, 1% and 0.5% of a minimum antibacterial dose.

Some examples of non-antibacterial daily doses of tetracycline compounds include about 20 mg/twice a day of doxycycline; about 38 mg of minocycline one, two, three or four times a day; and about 60 mg of tetracycline one, two, three or four times a day.

There is no necessary minimum effective amount of the tetracycline compound, as long as the amount administered is capable of providing an effective treatment of RAS. For example, when the amount is expressed as a percentage of the MIC plasma concentration, suitable minimum plasma concentrations include approximately 0.1%, 0.5%, 0.8% and 1% of the MIC plasma concentration. When the amount is expressed as a minimum actual plasma concentration, suitable actual plasma concentrations include approximately 0.01 μg/ml, 0.05 μg/ml, 0.1 μg/ml, 0.15 μg/ml, 0.2 μg/ml, 0.25 μg/ml and 0.3 μg/ml. When the dose is expressed as a percentage of a minimum antibacterial daily dose, the percentage is approximately 0.1%, 0.2%, 0.5%, 1%, 1.5% and 2% of the minimum antibacterial dose.

In a preferred embodiment, any form of doxycycline (e.g., doxycycline salts, such as doxycycline hyclate; and doxycycline hydrates, such as doxycycline monohydrate) is administered in a daily amount of, or equivalent to, from about 10 to about 60 milligrams of doxycycline, while maintaining a concentration in human plasma below the MIC.

In an especially preferred embodiment, doxycycline, a doxycycline salt, or a doxycycline hydrate, is administered at a dose of, or equivalent to, 20 milligram of doxycycline twice daily. Such a formulation is sold for the treatment of periodontal disease by CollaGenex Pharmaceuticals, Inc. of Newtown, Pa. under the trademark Periostat®.

Example 1 below summarizes a clinical study using 20 mg doxycycline hyclate tablets administered twice a day. A significant reduction in the number of ulcers and the pain associated with ulcers was observed.

In another embodiment, the tetracycline compound is a non-antibacterial tetracycline compound, such as the COLs discussed above. Since COLs have no, or substantially no, antibacterial activity, they can be administered at any effective dose at which side effects, if any, are acceptable. There is no risk of indiscriminate killing of bacteria, and the resulting threat of developing resistant bacteria.

For example, suitable maximum plasma concentrations of the COLs mentioned above include up to about 100 μg/ml, about 200 μg/ml and about 300 μg/ml. Suitable maximum daily doses of COLs include about 18 mg/kg/day, about 40 mg/kg/day, about 60 mg/kg/day and about 80 mg/kg/day.

A preferred COL is 6-demethyl-6-deoxy-4-de(dimethylamino)tetracycline (COL-3). COL-3 is administered in doses of up to about 200 mg/day, preferably about 150 mg/day, more preferably about 100 mg/day, or in amounts that result in plasma concentrations of up to about 50 μg/ml, about 40 μg/ml or about 30 μg/ml. For example, a dose of about 10 to about 20 mg/day of COL-3 produces plasma concentrations in humans of about 1.0 μg/ml.

There is no necessary minimum effective dose of COLs. Some typical minimum plasma concentrations of COLs include, for example, about 0.01 μg/ml, 0.1 μg/ml, 0.8 μg/ml, and 1.0 μg/ml. Some typical minimum daily doses of COLs include about 0.05 mg/day, about 0.1 mg/day, about 0.5 mg/day, about 1 mg/day, about 5 mg/day, or about 10 mg/day.

In a preferred embodiment, any of the doses of any of the tetracycline compounds mentioned above, e.g., doxycycline and COL-3, are administered by controlled release over a 24 hour period. For example, doxycycline is preferably administered in an amount of about 40 milligrams over the 24 hour period.

In another embodiment, the tetracycline compound is administered as a pharmaceutical composition comprising an active ingredient wherein the active ingredient consists essentially of a tetracycline compound in an amount that is effective to treat RAS but has substantially no antibacterial activity.

The actual preferred amounts of tetracycline compounds in a specified case will vary according to the particular compositions formulated, the mode of application, the particular sites of application, and the subject being treated (e.g., age, gender, size, tolerance to drug, etc.)

The tetracycline compounds can be in the form of pharmaceutically acceptable salts of the compounds. The term “pharmaceutically acceptable salt” refers to a salt prepared from a well-tolerated, nontoxic tetracycline compound and an acid or base. The acids may be inorganic or organic acids of tetracycline compounds. Examples of inorganic acids include hydrochloric, hydrobromic, nitric hydroiodic, sulfuric, and phosphoric acids. Examples of organic acids include carboxylic and sulfonic acids. The radical of the organic acids may be aliphatic or aromatic. Some examples of organic acids include formic, acetic, phenylacetic, propionic, succinic, glycolic, glucuronic, maleic, furoic, glutamic, benzoic, anthranilic, salicylic, phenylacetic, mandelic, embonic (pamoic), methanesulfonic, ethanesulfonic, panthenoic, benzenesulfonic, stearic, sulfanilic, alginic, tartaric, citric, gluconic, gulonic, arylsulfonic, and galacturonic acids. Appropriate organic bases may be selected, for example, from N,N-dibenzylethylenediamine, chloroprocaine, choline, diethanolamine, ethylenediamine, meglumine (N-methylglucamine), and procaine.

The tetracycline compounds of the invention are administered without administering a bisphosphonate compound. Bisphosphonates compounds are related to inorganic pyrophosphonic acid. The bisphosphonates include, as non-limiting examples, alendronate (4-amino-1-hydroxybutylidene bisphosphonic acid), clodronate (dichloromethane diphosphonic acid), etidronate (1-hydroxyethylidene diphosphanic acid) and pamidronate (3-amino-1-hydroxypropylidene bisphosphonic acid); also risedronate ([2-(3-pyridinyl)ethylidene]hydroxy bisphosphonic acid), tiludronate, i.e., tiludronic acid (4-chlorophenylthiomethylene bisphosphonic acid) and zolendronate.

Preferably, the tetracycline compounds have low phototoxicity, or are administered in an amount that results in a plasma level at which the phototoxicity is acceptable. Phototoxicity is a chemically-induced photosensitivity. Such photosensitivity renders skin susceptible to damage, e.g., sunburn, blisters, accelerated aging, erythemas and eczematoid lesions, upon exposure to light, in particular ultraviolet light. The preferred amount of the tetracycline compound produces no more phototoxicity than is produced by the administration of a 40 mg total daily dose of doxycycline.

Examples of tetracycline compounds with low phototoxicity include, but are not limited to, tetracycline compounds having general formulae:

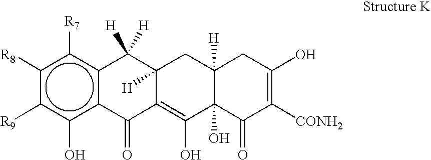

wherein: R7, R8, and R9 taken together in each case, have the following meanings:

| | | |
|---|---|---|
| R7 | R8 | R9 |

| | | | |
|---|---|---|---|
| hydrogen | hydrogen | amino | (COL-308) |
| hydrogen | hydrogen | palmitamide | (COL-311) |
| hydrogen | hydrogen | dimethylamino | (COL-306) |

and

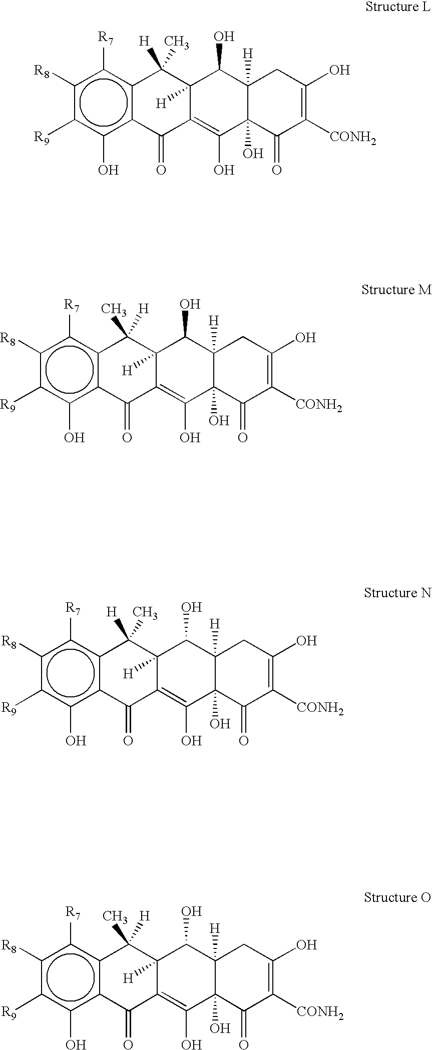

wherein: R7, R8, and R9 taken together in each case, have the following meanings:

| R7 | R8 | R9 | |
|---|---|---|---|
| hydrogen | hydrogen | acetamido | (COL-801) |
| hydrogen | hydrogen | dimethylaminoacetamido | (COL-802) |
| hydrogen | hydrogen | palmitamide | (COL-803) |
| hydrogen | hydrogen | nitro | (COL-804) |
| hydrogen | hydrogen | amino | (COL-805) |

and 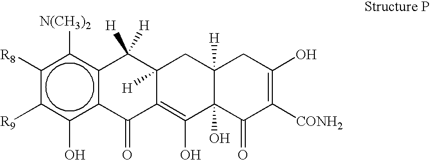
wherein: R8, and R9 taken together are, respectively, hydrogen and nitro (COL-1002).

The tetracycline compounds may be administered by methods known in the art. For example, the tetracycline compounds may be administered systemically. For the purposes of this specification, “systemic administration” means administration to a human by a method that causes the compounds to be absorbed into the bloodstream.

Preferably, the tetracycline compounds are administered orally by any method known in the art. For example, tetracyclines can be administered in the form of tablets, capsules, pills, troches, elixirs, suspensions, syrups, wafers, chewing gum and the like.

Additionally, the tetracycline compounds can be administered enterally or parenterally, e.g., intravenously; intramuscularly; subcutaneously, as injectable solutions or suspensions; intraperitoneally; or rectally. Administration can also be intranasally, in the form of, for example, an intranasal spray; or transdermally, in the form of, for example, a patch.

For the pharmaceutical purposes described above, the tetracycline compounds of the invention can be formulated per se in pharmaceutical preparations optionally with a suitable pharmaceutical carrier (vehicle) or excipient as understood by practitioners in the art. These preparations can be made according to conventional chemical methods.

In the case of tablets for oral use, carriers commonly used include lactose and corn starch, and lubricating agents such as magnesium stearate are commonly added. For oral administration in capsule form, useful carriers include lactose and corn starch. Further examples of carriers and excipients include milk, sugar, certain types of clay, gelatin, stearic acid or salts thereof, calcium stearate, talc, vegetable fats or oils, gums and glycols.

When aqueous suspensions are used for oral administration, emulsifying and/or suspending agents are commonly added. In addition, sweetening and/or flavoring agents may be added to the oral compositions.

For intramuscular, intraperitoneal, subcutaneous and intravenous use, sterile solutions of the tetracycline compounds can be employed, and the pH of the solutions can be suitably adjusted and buffered. For intravenous use, the total concentration of the solute(s) can be controlled in order to render the preparation isotonic.

The tetracycline compounds of the present invention can further comprise one or more pharmaceutically acceptable additional ingredient(s) such as alum, stabilizers, buffers, coloring agents, flavoring agents, and the like.

The tetracycline compounds may be administered at intervals. For example, the tetracycline compound may be administered 1-6 times a day, preferably 1-4 times a day.

The tetracycline compounds may be administered by controlled release. Controlled release administration is a method of drug delivery to achieve a certain level of the drug over a particular period of time. The level typically is measured by plasma concentration. Methods for controlled release of drugs are well known in the art. Further description of methods of delivering tetracycline compounds by controlled release can be found in international patent application PCT/US02/10748 (assigned to CollaGenex Pharmaceuticals, Inc. of Newtown, Pa.) which is incorporated herein by reference in its entirety.

In another embodiment, non-antibacterial tetracycline compounds can be administered topically to the oral mucosa. Particular non-antibacterial tetracycline compounds have only limited biodistribution, e.g., COL-5. In such cases, topical application is the preferred method of administration of the compound.

Carrier compositions deemed to be suited for topical use include gels, salves, lotions, creams, ointments, and the like. The non-antibacterial tetracycline compounds can also be incorporated into a support base, matrix, tissue adhesive, or the like which can be directly applied to the oral mucosa.

Topical application of the non-antibacterial tetracycline compounds are effective in treating RAS while not inducing significant toxicity in the human. For example, amounts of up to about 25% (w/w) in a vehicle are effective. Amounts of from about 0.1% to about 10% are preferred.

Combined or coordinated topical and systemic administration of the tetracycline compounds is also contemplated under the invention. For example, a non-absorbable non-antibacterial tetracycline compound can be administered topically, while a tetracycline compound capable of substantial absorption and effective systemic distribution in a human can be administered systemically.

The tetracycline compounds are prepared by methods known in the art. For example, natural tetracyclines may be modified without losing their antibacterial properties, although certain elements of the structure must be retained. The modifications that may and may not be made to the basic tetracycline structure have been reviewed by Mitscher in The Chemistry of Tetracyclines, Chapter 6, Marcel Dekker, Publishers, New York (1978). According to Mitscher, the substituents at positions 5-9 of the tetracycline ring system may be modified without the complete loss of antibacterial properties. Changes to the basic ring system or replacement of the substituents at positions 1-4 and 10-12, however, generally lead to synthetic tetracyclines with substantially less or effectively no antibacterial activity.

### EXAMPLE

The following example serves to provide further appreciation of the invention but is not meant in any way to restrict the effective scope of the invention.

### Clinical Study

The objective of this study was to evaluate the clinical effects of a 20 mg dose of doxycycline administered twice daily (i.e., a sub-antibacterial dose of doxycycline, “SDD”) vis-à-vis a placebo administered twice daily for the relief of symptoms of aphthous ulceration.

The study was a single center, placebo-controlled, double-bind, parallel group study. The study participants were fifty patients of an oral medicine clinic. The participants were screened to identify any known predisposing factors prior to the study. The participants had a history of minor aphthous ulceration that occurred more than two times per year.

The participants were administered SDD or placebo for three months. The participants were permitted to use any additional ulcer management therapies as needed. The participants kept daily diaries to record pain caused by ulcers using the Visual Analogue Scale (VAS), and to record the number of new ulcers each day. The VAS quantified pain over a continuum represented by a horizontal line 100 mm in length.

At the baseline, the oral soft tissues and ulcer count were evaluated in each of the participants. The ulcer history of each participant was taken. At the third month, oral soft tissues and ulcer count were reevaluated.

Of the 50 participants, 25 were administered SDD; and 25 were administered placebo. Of the 50 participants, 32 were females, 18 were males; and 49 were Caucasian, and one was Asian. Thirty-three participants completed the study. Of these participants, 16 were in the SDD group and 17 were in the placebo group.

As shown in Table I, SDD effectively treated RAS. Participants in the SDD group experienced less pain and had fewer ulcers vis-à-vis participants in the placebo group.

TABLE I

| | Placebo | SDD | |
|---|---|---|---|
| | Group | Group | P |
| Mean age | 43 | 37 | >0.05 |
| Mean years with ulcers (historic) | 15 | 15 | >0.05 |
| Mean number of ulcers per month | 8.0 | 5.8 | >0.05 |
| (historic) | | | |
| Mean number of ulcers present at | 1.4 | 1.7 | >0.05 |
| baseline | | | |
| Mean number of ulcers present at | 1.24 | 0.69 | >0.05 |
| month 3 | | | |
| Mean number of days using additional | 13.4 | 11.5 | >0.05 |
| treatments | | | |
| Mean total number of new ulcers in 3 | 35 | 17 | >0.05(0.07) |
| months | | | |
| Mean cumulative VAS (mm) | 1435 | 901 | >0.05 |

What is claimed is:

1. A method of treating recurrent aphthous stomatitis in a human in need thereof, the method comprising administering systemically to the human an antibacterial tetracycline compound in an amount that is effective to treat recurrent aphthous stomatitis but has substantially no antibacterial activity, without administering a bisphosphonate compound.

2. A method according to claim 1, wherein recurrent aphthous stomatitis is in the form of minor aphthae, major aphthae or herpetiform ulcers.

3. A method according to claim 1, wherein the antibacterial tetracycline compound is administered in an amount that results in a plasma concentration which is less than approximately 80%, 75%, 70%, 65%, 60%, 55%, 50%, 45%, 40%, 35%, 30%, 25%, 20%, 15%, 10%, 5%, 1% or 0.5% of MIC of the tetracycline compound for commonly-occurring bacteria.

4. A method according to claim 1, wherein the antibacterial tetracycline compound is administered in an amount which is less than approximately 80%, 75%, 70%, 65%, 60%, 55%, 50%, 45%, 40%, 35%, 30%, 25%, 20%, 15%, 10%, 5%, 1% or 0.5% of the minimum antibacterial dose.

5. A method according to claim 1, wherein the antibacterial tetracycline compound is doxycycline, minocycline, tetracycline, oxytetracycline, chlortetracycline, demeclocycline, lymecycline or pharmaceutically acceptable salts thereof.

6. A method according to claim 1, wherein the tetracycline compound is doxycycline administered in a daily amount of from about 10 to about 60 milligrams.

7. A method according to claim 6, wherein the doxycycline is administered twice a day in a dose of about 20 mg.

8. A method according to claim 6, wherein the doxycycline is administered by controlled release over a 24 hour period.

9. A method according to claim 8, wherein the doxycycline is administered in an amount of about 40 milligrams.

10. A method according to claim 1, wherein the tetracycline compound is minocycline.

11. A method according to claim 1, wherein the tetracycline compound is tetracycline.

12. A method according to claim 1, wherein the tetracycline compound is doxycycline administered in an amount which results in a plasma concentration in the range of about 0.1 to about 0.8 μg/ml.

13. A method according to claim 1, wherein the systemic administration is oral administration or intravenous injection.

14. A method of treating recurrent aphthous stomatitis in a human in need thereof, the method comprising administering to the human a non-antibacterial tetracycline compound in an amount that is effective to treat recurrent aphthous stomatitis but has substantially no antibacterial activity, without administering a bisphosphonate compound.

15. A method according to claim 14, wherein the non-antibacterial tetracycline compound is: 4-de(dimethylamino)tetracycline, tetracyclinonitrile, 6-demethyl-6-deoxy-4-de(dimethylamino)tetracycline, 4-de(dimethylamino)-7-chlorotetracycline, tetracycline pyrazole, 4-hydroxy-4-de(dimethylamino)tetracycline, 4-de(dimethylamino)-12α-deoxytetracycline, 6-α-deoxy-5-hydroxy-4-de(dimethylamino)tetracycline, 4-de(dimethylamino)-12α-deoxyanhydrotetracycline, or 4-de(dimethylamino)minocycline.

16. A method according to claim 15, wherein the non-antibacterial tetracycline compound is 6-demethyl-6-deoxy-4-de(dimethylamino)tetracycline.

17. A method according to claim 14, wherein the tetracycline compound is selected from the group consisting of: 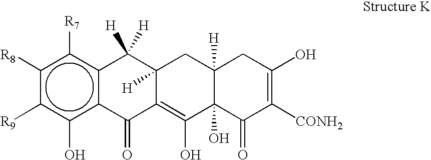
wherein R7, R8, and R9 taken together in each case, have the following meanings:

| R7 | R8 | R9 |
|---|---|---|
| hydrogen | hydrogen | amino |
| hydrogen | hydrogen | palmitamide |
| hydrogen | hydrogen | dimethylamino |

and 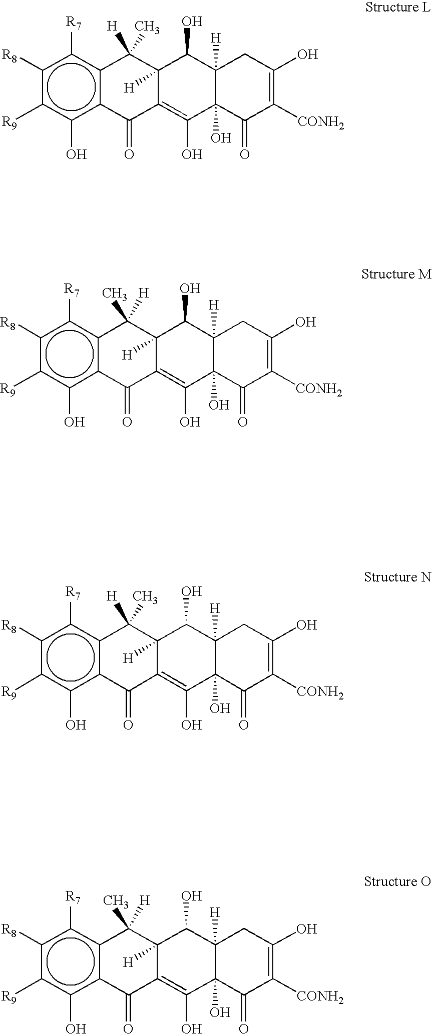
wherein R7, R8, and R9 taken together in each case, have the following meanings:

| R7 | R8 | R9 |
|---|---|---|
| hydrogen | hydrogen | acetamido |
| hydrogen | hydrogen | dimethylaminoacetamido |
| hydrogen | hydrogen | nitro |
| hydrogen | hydrogen | amino |
| hydrogen | hydrogen | palmitamide |

and 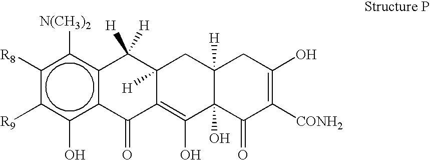
wherein R8, and R9 taken together are, respectively, hydrogen and nitro.

18. A method according to claim 14, wherein the administration is systemic administration.

19. A method according to claim 18, wherein the systemic administration is oral administration, intravenous injection, intramuscular injection, subcutaneous administration, transdermal administration or intranasal administration.

20. A method according to claim 14, wherein the administration is topical administration.

21. A method of treating recurrent aphthous stomatitis in a human in need thereof, the method comprising administering systemically to the human a pharmaceutical composition comprising an active ingredient wherein the active ingredient consists essentially of a tetracycline compound in an amount that is effective to treat recurrent aphthous stomatitis but has substantially no antibacterial activity, without administering a bisphosphonate compound.
